(19) United States
(12) Patent Application Publication
(10) Pub. No.: US 2026/0069318 A1
(43) Pub. Date: Mar. 12, 2026

(54) BONE IMPLANT POSITIONING CONFIRMATION SYSTEMS AND METHODS

(71) Applicant: Proprio, Inc., Seattle, WA (US)

(72) Inventors: David Lee Fiorella, Seattle, WA (US); Camille Cheli Farley, Kirkland, WA (US); Nicholas Matthew Miclette, Seattle, WA (US); Thomas A. Carls, Seattle, WA (US)

(21) Appl. No.: 19/316,583

(22) Filed: Sep. 2, 2025

Related U.S. Application Data
(60) Provisional application No. 63/691,929, filed on Sep. 6, 2024.

(51) Int. Cl. A61B 17/70; A61B 34/00; A61B 34/10; A61B 34/20; G06T 7/70

(52) U.S. Cl. CPC A61B 17/7001; A61B 34/10; A61B 34/20; A61B 34/25; G06T 7/70; A61B 2034/102; A61B 2034/2057; G06T 2207/10052; G06T 2207/30012; G06T 2207/30052

(57) ABSTRACT

Methods of determining the position and orientation of an implant within a bone structure during a surgical procedure utilizing intraoperative data collected without radiation-based imaging, and associated systems and devices are disclosed herein. In some embodiments, a representative method includes capturing intraoperative data of the implant and the bone structure, wherein the bone structure has a portion external to the bone structure and visible in the intraoperative data. The method can further include registering the visible portion of the implant in the intraoperative data to (i) a three-dimensional model of the implant and (ii) initial image data of the bone structure. The method can further include determining the position and orientation of the implant relative to the bone structure based on both registrations.

### CROSS-REFERENCE TO RELATED APPLICATION

This application claims the benefit of U.S. Provisional Ser. No. 63/691,929, filed Sep. 6, 2024, and titled “BONE IMPLANT POSITIONING CONFIRMATION SYSTEMS AND METHODS,” which is incorporated herein by reference in its entirety.

### TECHNICAL FIELD

The present technology generally relates to methods, systems, and devices for determining the position and orientation of an implant relative to a bone structure during a surgical procedure, such as a spinal surgical procedure.

### BACKGROUND

In many orthopedic procedures, implants are secured in bone for a variety of functions, such as fixing or holding bones in specific positions, providing attachment points for additional structures, and/or providing structural support. Due to the density of some bones and proximity to other anatomical systems, major challenges with securing medical implants into bone include safely delivering the implant and verifying its proper positioning within the bone. Depending on the bone and other factors (e.g., implantation depth), visual confirmation of the position of the implant may be difficult or impossible. Thus, surgeons typically rely on conventional imaging techniques to confirm implant position, such as computerized tomography (CT) imaging, X-ray imaging, and/or the like.

However, such conventional imaging techniques expose the patient and healthcare team to radiation and can be cumbersome and time-consuming. Accordingly, imaging to confirm implant positioning is often performed late during the surgical procedure (e.g., after multiple implants have been positioned) or even post-operatively to avoid additional radiation exposure and to not unduly slow the surgical procedure. For example, during some spinal surgical procedures, multiple pedicle screws are secured to one or more vertebrae before a C-arm machine is brought in and used to confirm their proper positioning. If the imaging indicates that a pedicle screw is improperly positioned, it can be difficult and time consuming to go back and fix the positioning. Thus, solutions are needed that can ensure proper positioning of bone implants without undesirable doses of radiation and/or costly rework.

### BRIEF DESCRIPTION OF THE DRAWINGS

Many aspects of the present disclosure can be better understood with reference to the following drawings. The components in the drawings are not necessarily to scale. Instead, emphasis is placed on clearly illustrating the principles of the present disclosure.

FIG. 1 is a schematic view of an imaging system in accordance with embodiments of the present technology.

FIG. 2 is a perspective view of an environment employing the imaging system of FIG. 1 in accordance with embodiments of the present technology.

FIG. 3 is an isometric view of a portion of the imaging system of FIG. 1 illustrating four cameras of a sensor array of the imaging system in accordance with embodiments of the present technology.

FIG. 4 is a flow diagram of a process or method for determining a position and orientation of an implant relative to a bone structure in which it is implanted in accordance with embodiments of the present technology.

FIG. 5A-5B are side views of a vertebra of a patient and a pedicle screw before and after implantation of the pedicle screw in the vertebra, respectively, in accordance with embodiments of the present technology.

FIG. 6A-6C are an axial, sagittal, and coronal cutaway view, respectively, of initial image data of the vertebra of FIGS. 5A and 5B illustrating the overlay of a 3D model of the pedicle screw of FIGS. 5A and 5B in accordance with embodiments of the present technology.

### DETAILED DESCRIPTION

Aspects of the present technology are directed generally to methods of determining the position and orientation of an implant within a bone structure during a surgical procedure utilizing intraoperative data collected without radiation-based imaging, and associated systems and devices. In some embodiments, a representative method of determining a position and orientation of an implant secured within a bone structure during a surgical procedure includes capturing intraoperative data of the implant and the bone structure. The intraoperative data can include light field image data of the bone structure and the implant, RGB data of the bone structure and the implant, depth data (e.g., a point cloud, mesh, and/or other three-dimensional (3D) data set) of the bone structure and the implant, and/or the like. After implantation, the implant may be positioned within the bone structure such that it has a first portion external to the bone structure and visible in the intraoperative data (and to the surgical team) and a second portion positioned within the bone structure and thereby not visible in the intraoperative data. The method can further include registering the visible portion of the implant in the intraoperative data to a 3D model of the implant. The 3D model can be a computer-aided design (CAD) model of the implant that provides volumetric data of the implant. The method can further include registering the visible portion of the implant in the intraoperative data to initial image data of the bone structure, such as computed-tomography (CT) scan data of the bone structure taken preoperatively. The method can further include determining the position and orientation of the implant relative to the bone structure based on the registrations of the visible portion of the implant in the intraoperative data to (i) the 3D model of the implant and (ii) the initial image data of the bone structure. That is, for example, the method can align the 3D model with the initial image data based on the registrations to provide volumetric information about the position and orientation of the entire implant (including the second portion positioned within and thereby obscured by the bone structure) based on the intraoperatively-captured data of the visible portion of the implant.

In some aspects of the present technology, the present technology can accurately, reliably, and quickly determine the position and orientation of a bone implant without the need for radiation-based imaging techniques. In contrast, conventional surgical procedures and techniques typically determine implant position and orientation through one or more CT scans and/or other X-ray imaging techniques late in the surgical procedure, or post-operatively. However, such conventional imaging techniques expose the patient and healthcare team to radiation and can be cumbersome and time-consuming. Accordingly, some aspects of the present technology can avoid these problems by providing an immediate and accurate assessment of the position of an implant within a bone structure, allowing medical personnel to make safety and efficacy assessments without requiring cumbersome and time-consuming radiation-based imaging. Likewise, the present technology can provide immediate feedback about the properness of implant placement, rather than requiring medical personnel to discover an improperly positioned implant late in a procedure or post-operatively.

In some embodiments, the method can be repeated for one or more additional implants to be implanted within the bone structure during the surgical procedure. For example, the surgical procedure can be a spinal surgical procedure and the bone structure can comprise a spine. The implant(s) can be pedicle screws that are driven into various vertebrae of the spine. Each of the multiple pedicle screws can be tracked continuously or near-continuously during the surgical procedure as they are implanted to provide an indication of the position and orientation of the pedicle screw relative to the vertebrae—including the position and orientation of a visibly-obscured portion of the pedicle screw positioned within bone of the vertebrae—to help a surgeon position and/or reposition the pedicle screws over the course of the procedure.

In some embodiments, the method further includes capturing multiple images of the implant within the bone structure over the course of a procedure and continuously updating the registration of the visible portion of the implant to the initial image model of the bone structure and the 3D model of the implant to detect a loss of fixation of the implant's initial position with the bone structure. For example, a surgeon can determine that the 3D model of the implant is moving in an undesired manner relative to the initial image data indicative of a loss of fixation of the implant's initial position with the bone structure as the implant is torqued/loaded during the surgical procedure. For example, such a method can determine that one or more pedicle screws are moving (e.g., ploughing) through a spine as rods are connected to the pedicle screws and loaded and/or as the spine is manipulated to achieve a desired alignment during the surgical procedure.

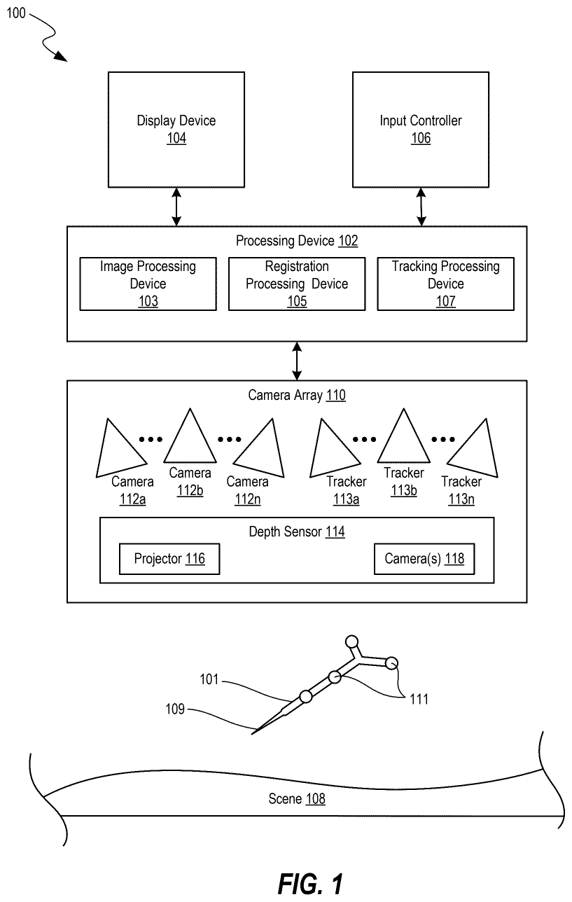

Specific details of several embodiments of the present technology are described herein with reference to FIG. 1-6. The present technology, however, can be practiced without some of these specific details. In some instances, well-known structures and techniques often associated with sensor arrays, RGB imaging, light field imaging, depth sensing, 3D models, registration processes, and the like have not been shown in detail so as not to obscure the present technology.

The terminology used in the description presented below is intended to be interpreted in its broadest reasonable manner, even though it is being used in conjunction with a detailed description of certain specific embodiments of the disclosure. Certain terms can even be emphasized below; however, any terminology intended to be interpreted in any restricted manner will be overtly and specifically defined as such in this Detailed Description section. Moreover, although frequently described in the context of determining the position and orientation of a pedicle screw during a spinal surgical procedure, the present technology can be used to determine the position and orientation of other implants during other surgical procedures.

The accompanying Figures depict embodiments of the present technology and are not intended to be limiting of its scope. Depicted elements are not necessarily drawn to scale, and various elements can be arbitrarily enlarged to improve legibility. Component details can be abstracted in the figures to exclude details as such details are unnecessary for a complete understanding of how to make and use the present technology. Many of the details, dimensions, angles, and other features shown in the Figures are merely illustrative of particular embodiments of the disclosure. Accordingly, other embodiments can have other dimensions, angles, and features without departing from the spirit or scope of the present technology.

The headings provided herein are for convenience only and should not be construed as limiting the subject matter disclosed. To the extent any materials incorporated herein by reference conflict with the present disclosure, the present disclosure controls.

### I. Selected Embodiments of Imaging Systems

FIG. 1 is a schematic view of an imaging system 100 (“system 100”) in accordance with embodiments of the present technology. In some embodiments, the system 100 can be a synthetic augmented reality system, a virtual-reality imaging system, an augmented-reality imaging system, a mediated-reality imaging system, and/or a non-immersive computational imaging system. In the illustrated embodiment, the system 100 includes a processing device 102 that is communicatively coupled to one or more display devices 104, one or more input controllers 106, and a sensor array 110 (e.g., a camera array, a sensor head, and/or the like). In other embodiments, the system 100 can comprise additional, fewer, or different components. In some embodiments, the system 100 includes some features that are generally similar or identical to those of the mediated-reality imaging systems disclosed in (i) U.S. patent application Ser. No. 16/586,375, filed Sep. 27, 2019, and titled “CAMERA ARRAY FOR A MEDIATED-REALITY SYSTEM,” and/or (ii) U.S. patent application Ser. No. 15/930,305, filed May 12, 2020, and titled “METHODS AND SYSTEMS FOR IMAGING A SCENE, SUCH AS A MEDICAL SCENE, AND TRACKING OBJECTS WITHIN THE SCENE,” each of which is incorporated herein by reference in its entirety.

In the illustrated embodiment, the sensor array 110 includes a plurality of cameras 112 (identified individually as cameras 112a-112n; which can also be referred to as first cameras) that can each capture images of a scene 108 (e.g., first image data) from a different perspective. The scene 108 can include for example, a patient undergoing surgery (e.g., spinal surgery) and/or another medical procedure. In other embodiments, the scene 108 can be another type of scene. The sensor array 110 can further include dedicated object tracking hardware 113 (e.g., including individually identified trackers 113a-113n) that captures positional data of one more objects, such as an instrument 101 (e.g., a surgical instrument or tool) having a tip 109, to track the movement and/or orientation of the objects through/in the scene 108. In some embodiments, the cameras 112 and the trackers 113 are positioned at fixed locations and orientations (e.g., poses) relative to one another. For example, the cameras 112 and the trackers 113 can be structurally secured by/to a mounting structure (e.g., a common frame) at predefined fixed locations and orientations. In some embodiments, the cameras 112 are positioned such that neighboring cameras 112 share overlapping views of the scene 108. In general, the position of the cameras 112 can be selected to maximize clear and accurate capture of all or a selected portion of the scene 108. Likewise, the trackers 113 can be positioned such that neighboring trackers 113 share overlapping views of the scene 108. Therefore, all or a subset of the cameras 112 and the trackers 113 can have different extrinsic parameters, such as position and orientation (e.g., pose).

In some embodiments, the cameras 112 in the sensor array 110 are synchronized to capture images of the scene 108 simultaneously (within a threshold temporal error). In some embodiments, all or a subset of the cameras 112 are light field, plenoptic, and/or RGB cameras that capture information about the light field emanating from the scene 108 (e.g., information about the intensity of light rays in the scene 108 and also information about a direction the light rays are traveling through space). In some embodiments, image data from the cameras 112 can be used to reconstruct a light field of the scene 108. More specifically, the cameras 112 can be RGB cameras that capture a combined image data set for reconstructing a light field of the scene 108. Therefore, in some embodiments the images captured by the cameras 112 encode depth information representing a surface geometry of the scene 108. In some embodiments, the cameras 112 are substantially identical. In other embodiments, the cameras 112 include multiple cameras of different types. For example, different subsets of the cameras 112 can have different intrinsic parameters such as focal length, sensor type, optical components, and the like. The cameras 112 can have charge-coupled device (CCD) and/or complementary metal-oxide semiconductor (CMOS) image sensors and associated optics. Such optics can include a variety of configurations including lensed or bare individual image sensors in combination with larger macro lenses, micro-lens arrays, prisms, and/or negative lenses. For example, the cameras 112 can be separate light field cameras each having their own image sensors and optics. In other embodiments, some or all of the cameras 112 can comprise separate microlenslets (e.g., lenslets, lenses, microlenses) of a microlens array (MLA) that share a common image sensor. In other embodiments, some or all of the cameras 112 can be RGB (e.g., color) cameras having visible imaging sensors that together provide a light field data set of the scene 108.

In some embodiments, the trackers 113 are imaging devices, such as infrared (IR) cameras that can capture images of the scene 108 from a different perspective compared to other ones of the trackers 113. Accordingly, the trackers 113 and the cameras 112 can have different spectral sensitives (e.g., infrared vs. visible wavelength). In some embodiments, the trackers 113 capture image data of a plurality of optical markers (e.g., fiducial markers, marker balls) in the scene 108, such as markers 111 coupled to the instrument 101.

In the illustrated embodiment, the sensor array 110 further includes a depth sensor 114. In some embodiments, the depth sensor 114 includes (i) one or more projectors 116 that project a structured light pattern onto/into the scene 108 and (ii) one or more depth cameras 118 (which can also be referred to as second cameras) that capture second image data of the scene 108 including the structured light projected onto the scene 108 by the projector 116. The projector 116 can project a speckled pattern or a pattern of dots, for example. The projector 116 and the depth cameras 118 can operate in the same wavelength and, in some embodiments, can operate in a wavelength different than the cameras 112. For example, the cameras 112 can capture the first image data in the visible spectrum, while the depth cameras 118 capture the second image data in the infrared spectrum. In some embodiments, the depth cameras 118 have a resolution that is less than a resolution of the cameras 112. For example, the depth cameras 118 can have a resolution that is less than 70%, 60%, 50%, 40%, 30%, or 20% of the resolution of the cameras 112. In other embodiments, the depth sensor 114 can include other types of dedicated depth detection hardware (e.g., a LiDAR detector) for determining the surface geometry of the scene 108. In other embodiments, the sensor array 110 can omit the projector 116 and/or the depth cameras 118.

In the illustrated embodiment, the processing device 102 includes an image processing device 103 (e.g., an image processor, an image processing module, an image processing unit), a registration processing device 105 (e.g., a registration processor, a registration processing module, a registration processing unit), and a tracking processing device 107 (e.g., a tracking processor, a tracking processing module, a tracking processing unit). The image processing device 103 can (i) receive the first image data captured by the cameras 112 (e.g., light field images, light field image data, RGB images) and depth information from the depth sensor 114 (e.g., the second image data captured by the depth cameras 118), and (ii) process the image data and depth information to synthesize (e.g., generate, reconstruct, render) a three-dimensional (3D) output image of the scene 108 corresponding to a virtual camera perspective (e.g., a novel camera perspective). The output image can correspond to an approximation of an image of the scene 108 that would be captured by a camera placed at an arbitrary position and orientation corresponding to the virtual camera perspective. In some embodiments, the image processing device 103 can further receive and/or store calibration data for the cameras 112 and/or the depth cameras 118 and synthesize the output image based on the image data, the depth information, and/or the calibration data. More specifically, the depth information and the calibration data can be used/combined with the images from the cameras 112 to synthesize the output image as a 3D (or stereoscopic 2D) rendering of the scene 108 as viewed from the virtual camera perspective. In some embodiments, the image processing device 103 can synthesize the output image using any of the methods disclosed in U.S. patent application Ser. No. 16/457,780, filed Jun. 28, 2019, and titled “SYNTHESIZING AN IMAGE FROM A VIRTUAL PERSPECTIVE USING PIXELS FROM A PHYSICAL IMAGER ARRAY WEIGHTED BASED ON DEPTH ERROR SENSITIVITY,” which is incorporated herein by reference in its entirety. In other embodiments, the image processing device 103 can generate the virtual camera perspective based only on the images captured by the cameras 112—without utilizing depth information from the depth sensor 114. For example, the image processing device 103 can generate the virtual camera perspective by interpolating between the different images captured by one or more of the cameras 112. In some embodiments the image processing device 103 can utilize a neural radiance field (NeRF) rendering algorithm to synthesize and render an output image of the scene 108 based on RGB images captured by the cameras 112 and depth data captured by the depth sensor 114.

The image processing device 103 can synthesize the output image from images captured by a subset (e.g., two or more) of the cameras 112 in the sensor array 110, and does not necessarily utilize images from all of the cameras 112. For example, for a given virtual camera perspective, the processing device 102 can select a stereoscopic pair of images from two of the cameras 112. In some embodiments, such a stereoscopic pair can be selected to be positioned and oriented to most closely match the virtual camera perspective. In some embodiments, the image processing device 103 (and/or the depth sensor 114) estimates a depth for each surface point of the scene 108 relative to a common origin to generate a point cloud and/or a 3D mesh that represents the surface geometry of the scene 108. Such a representation of the surface geometry can be referred to as a surface reconstruction, a 3D reconstruction, a 3D surface reconstruction, a depth map, a depth surface, and/or the like. In some embodiments, the depth cameras 118 of the depth sensor 114 detect the structured light projected onto the scene 108 by the projector 116 to estimate depth information of the scene 108. In some embodiments, the image processing device 103 estimates depth from multiview image data from the cameras 112 using techniques such as light field correspondence, stereo block matching, photometric symmetry, correspondence, defocus, block matching, texture-assisted block matching, structured light, and the like, with or without utilizing information collected by the depth sensor 114. In other embodiments, depth may be acquired by a specialized set of the cameras 112 performing the aforementioned methods in another wavelength. In some embodiments, the image processing device 103 can generate a stereoscopic view by selecting images from a pair of the cameras 112 using any of the methods disclosed in U.S. patent application Ser. No. 17/521,235, filed Nov. 11, 2021, and titled “METHODS FOR GENERATING STEREOSCOPIC VIEWS IN MULTICAMERA SYSTEMS, AND ASSOCIATED DEVICES AND SYSTEMS,” which is incorporated herein by reference in its entirety.

In some embodiments, the registration processing device 105 receives and/or stores initial image data, such as image data of a three-dimensional volume of a patient (3D image data). The image data can include, for example, computerized tomography (CT) scan data, magnetic resonance imaging (MRI) scan data, ultrasound images, fluoroscope images, and/or other medical or other image data. The image data can be segmented or unsegmented. The registration processing device 105 can register the initial image data to the real-time images captured by the cameras 112 and/or the depth sensor 114 by, for example, determining one or more transforms/transformations/mappings between the two. The processing device 102 (e.g., the image processing device 103) can then apply the one or more transformations to the initial image data such that the initial image data can be aligned with (e.g., overlaid on) the output image of the scene 108 in real-time or near real-time on a frame-by-frame basis, even as the virtual perspective changes. That is, the image processing device 103 can fuse the initial image data with the real-time output image of the scene 108 to present a mediated-reality view that enables, for example, a surgeon to simultaneously view a surgical site in the scene 108 and the underlying 3D anatomy of a patient undergoing an operation. In some embodiments, the registration processing device 105 can register the initial image data to the real-time images by using any of the methods disclosed in U.S. patent application Ser. No. 17/140,885, filed Jan. 4, 2021, and titled “METHODS AND SYSTEMS FOR REGISTERING PREOPERATIVE IMAGE DATA TO INTRAOPERATIVE IMAGE DATA OF A SCENE, SUCH AS A SURGICAL SCENE,” and/or U.S. patent application Ser. No. 18/084,389, filed Dec. 19, 2022, and titled “METHODS AND SYSTEMS FOR REGISTERING PREOPERATIVE IMAGE DATA TO INTRAOPERATIVE IMAGE DATA OF A SCENE, SUCH AS A SURGICAL SCENE,” each of which is incorporated by reference herein in its entirety. In some embodiments, the registration processing device 105 can register the initial image data to the real-time images using depth information from the depth sensor 114, using X-ray data and/or other medical imaging data, using tracing information from an instrument moved through the scene 108, and/or using other data.

In some embodiments, the tracking processing device 107 processes positional data captured by the trackers 113 to track objects (e.g., the instrument 101) within the vicinity of the scene 108. For example, the tracking processing device 107 can determine the position of the markers 111 in the 2D images captured by two or more of the trackers 113, and can compute the 3D position of the markers 111 via triangulation of the 2D positional data. More specifically, in some embodiments the trackers 113 include dedicated processing hardware for determining positional data from captured images, such as a centroid of the markers 111 in the captured images. The trackers 113 can then transmit the positional data to the tracking processing device 107 for determining the 3D position of the markers 111. In other embodiments, the tracking processing device 107 can receive the raw image data from the trackers 113. In a surgical application, for example, the tracked object can comprise a surgical instrument, an implant, a hand or arm of a physician or assistant, and/or another object having the markers 111 mounted thereto. In some embodiments, the processing device 102 can recognize the tracked object as being separate from the scene 108, and can apply a visual effect to the 3D output image to distinguish the tracked object by, for example, highlighting the object, labeling the object, and/or applying a transparency to the object.

In some embodiments, functions attributed to the processing device 102, the image processing device 103, the registration processing device 105, and/or the tracking processing device 107 can be practically implemented by two or more physical devices. For example, in some embodiments a synchronization controller (not shown) controls images displayed by the projector 116 and sends synchronization signals to the cameras 112 to ensure synchronization between the cameras 112 and the projector 116 to enable fast, multi-frame, multicamera structured light scans. Additionally, such a synchronization controller can operate as a parameter server that stores hardware specific configurations such as parameters of the structured light scan, camera settings, and camera calibration data specific to the camera configuration of the sensor array 110. The synchronization controller can be implemented in a separate physical device from a display controller that controls the display device 104, or the devices can be integrated together.

The processing device 102 can comprise a processor and a non-transitory computer-readable storage medium that stores instructions that when executed by the processor, carry out the functions attributed to the processing device 102 as described herein. Although not required, aspects and embodiments of the present technology can be described in the general context of computer-executable instructions, such as routines executed by a general-purpose computer, e.g., a server or personal computer. Those skilled in the relevant art will appreciate that the present technology can be practiced with other computer system configurations, including Internet appliances, hand-held devices, wearable computers, cellular or mobile phones, multi-processor systems, microprocessor-based or programmable consumer electronics, set-top boxes, network PCs, mini-computers, mainframe computers and the like. The present technology can be embodied in a special purpose computer or data processor that is specifically programmed, configured or constructed to perform one or more of the computer-executable instructions explained in detail below. Indeed, the term “computer” (and like terms), as used generally herein, refers to any of the above devices, as well as any data processor or any device capable of communicating with a network, including consumer electronic goods such as game devices, cameras, or other electronic devices having a processor and other components, e.g., network communication circuitry.

The present technology can also be practiced in distributed computing environments, where tasks or modules are performed by remote processing devices, which are linked through a communications network, such as a Local Area Network (“LAN”), Wide Area Network (“WAN”), or the Internet. In a distributed computing environment, program modules or sub-routines can be located in both local and remote memory storage devices. Aspects of the present technology described below can be stored or distributed on computer-readable media, including magnetic and optically readable and removable computer discs, stored as in chips (e.g., EEPROM or flash memory chips). Alternatively, aspects of the present technology can be distributed electronically over the Internet or over other networks (including wireless networks). Those skilled in the relevant art will recognize that portions of the present technology can reside on a server computer, while corresponding portions reside on a client computer. Data structures and transmission of data particular to aspects of the present technology are also encompassed within the scope of the present technology.

The virtual camera perspective is controlled by an input controller 106 that can update the virtual camera perspective based on user driven changes to the camera's position and rotation. The output images corresponding to the virtual camera perspective can be outputted to the display device 104. In some embodiments, the image processing device 103 can vary the perspective, the depth of field (e.g., aperture), the focus plane, and/or another parameter of the virtual camera (e.g., based on an input from the input controller) to generate different 3D output images without physically moving the sensor array 110. The display device 104 can receive output images (e.g., the synthesized 3D rendering of the scene 108) and display the output images for viewing by one or more viewers. In some embodiments, the processing device 102 receives and processes inputs from the input controller 106 and processes the captured images from the sensor array 110 to generate output images corresponding to the virtual perspective in substantially real-time or near real-time as perceived by a viewer of the display device 104 (e.g., at least as fast as the frame rate of the sensor array 110).

Additionally, the display device 104 can display a graphical representation on/in the image of the virtual perspective of any (i) tracked objects within the scene 108 (e.g., a surgical instrument) and/or (ii) registered or unregistered initial image data. That is, for example, the system 100 (e.g., via the display device 104) can blend augmented data into the scene 108 by overlaying and aligning information on top of “passthrough” images of the scene 108 captured by the cameras 112 and/or generated by images captured by the cameras 112. Moreover, the system 100 can create a mediated-reality experience where the scene 108 is reconstructed using light field image data of the scene 108 captured by the cameras 112, and where instruments are virtually represented in the reconstructed scene via information from the trackers 113. Additionally or alternatively, the system 100 can remove the original scene 108 and completely replace it with a registered and representative arrangement of the initial image data, thereby removing information in the scene 108 that is not pertinent to a user's task.

The display device 104 can comprise, for example, a head-mounted display device, a monitor, a computer display, and/or another display device. In some embodiments, the input controller 106 and the display device 104 are integrated into a head-mounted display device and the input controller 106 comprises a motion sensor that detects position and orientation of the head-mounted display device. In some embodiments, the system 100 can further include a separate tracking system (not shown), such an optical tracking system, for tracking the display device 104, the instrument 101, and/or other components within the scene 108. Such a tracking system can detect a position of the head-mounted display device 104 and input the position to the input controller 106. The virtual camera perspective can then be derived to correspond to the position and orientation of the head-mounted display device 104 in the same reference frame and at the calculated depth (e.g., as calculated by the depth sensor 114) such that the virtual perspective corresponds to a perspective that would be seen by a viewer wearing the head-mounted display device 104. Thus, in such embodiments the head-mounted display device 104 can provide a real-time rendering of the scene 108 as it would be seen by an observer without the head-mounted display device 104. Alternatively, the input controller 106 can comprise a user-controlled control device (e.g., a mouse, pointing device, handheld controller, gesture recognition controller) that enables a viewer to manually control the virtual perspective displayed by the display device 104.

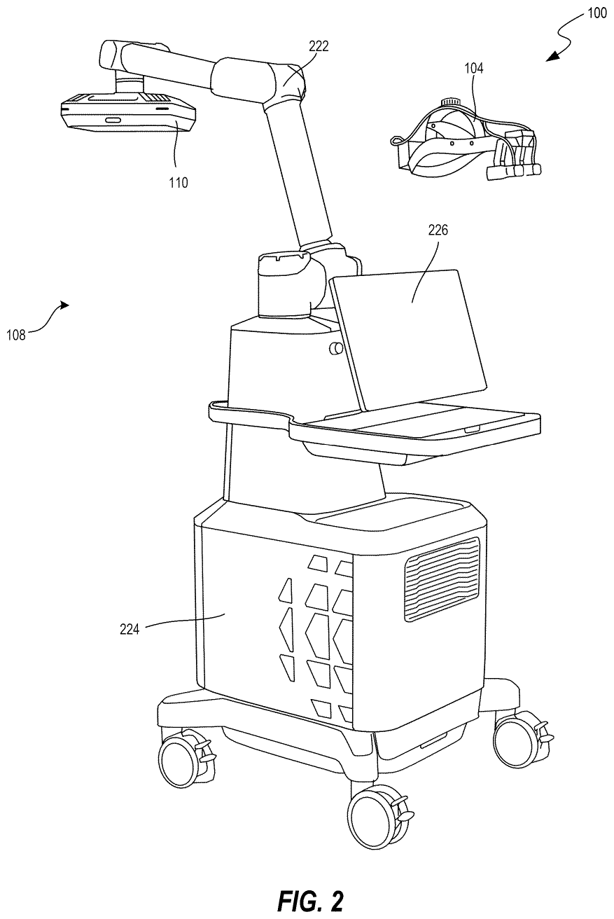

FIG. 2 is a perspective view of an environment (e.g., a surgical environment) employing the system 100 (e.g., for a surgical application) in accordance with embodiments of the present technology. In the illustrated embodiment, the sensor array 110 is positioned over the scene 108 (e.g., a surgical site) and supported/positioned via a mover 222 that is operably coupled to a workstation 224. In some embodiments, the mover 222 is manually movable to position the sensor array 110 while, in other embodiments, the mover 222 is robotically controlled in response to the input controller 106 (FIG. 1) and/or another controller. Accordingly, the mover 222 can be referred to as a robotic mover, a robotic arm, a robotically-controlled arm, and/or the like. The mover 222 allows the sensor array 110 to be precisely moved relative to the scene 108 such that the sensor array 110 is mobile relative to the scene 108.

In the illustrated embodiment, the display device 104 is a head-mounted display device (e.g., a virtual reality headset, augmented reality headset). The workstation 224 can include a computer to control various functions of the processing device 102, the display device 104, the input controller 106, the sensor array 110, and/or other components of the system 100 shown in FIG. 1. Accordingly, in some embodiments the processing device 102 and the input controller 106 are each integrated in the workstation 224. In some embodiments, the workstation 224 includes a secondary display 226 that can display a user interface for performing various configuration functions, a mirrored image of the display on the display device 104, and/or other useful visual images/indications. In other embodiments, the system 100 can include more or fewer display devices. For example, in addition to (or alternatively to) the display device 104 and the secondary display 226, the system 100 can include another display (e.g., a medical grade computer monitor) visible to the user wearing the display device 104.

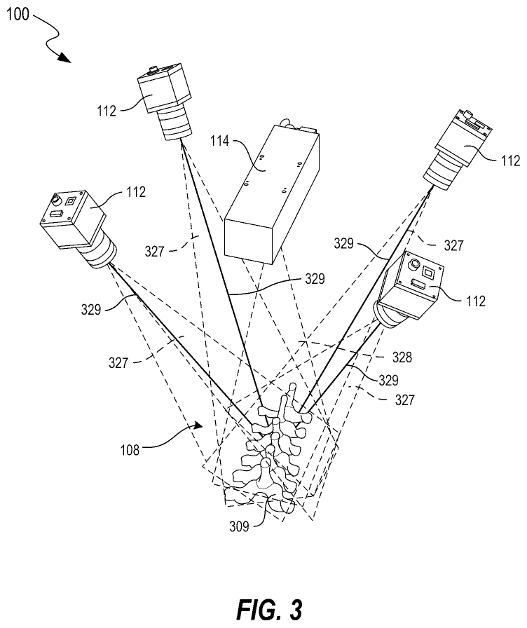

FIG. 3 is an isometric view of a portion of the system 100 illustrating four of the cameras 112 in accordance with embodiments of the present technology. Other components of the system 100 (e.g., other portions of the sensor array 110, the processing device 102, etc.) are not shown in FIG. 3 for the sake of clarity. In the illustrated embodiment, each of the cameras 112 has a field of view 327 and a focal axis 329. Likewise, the depth sensor 114 can have a field of view 328 aligned with a portion of the scene 108. The cameras 112 can be oriented such that the fields of view 327 are aligned with a portion of the scene 108 and at least partially overlap one another to together define an imaging volume. In some embodiments, some or all of the field of views 327, 328 at least partially overlap. For example, in the illustrated embodiment the fields of view 327, 328 converge toward a common measurement volume including a portion of a spine 309 of a patient (e.g., a human patient) located in/at the scene 108. In some embodiments, the cameras 112 are further oriented such that the focal axes 329 converge to a common point in the scene 108. In some aspects of the present technology, the convergence/alignment of the focal axes 329 can generally maximize disparity measurements between the cameras 112. In some embodiments, the cameras 112 and the depth sensor 114 are fixedly positioned relative to one another (e.g., rigidly mounted to a common frame) such that a relative positioning of the cameras 112 and the depth sensor 114 relative to one another is known and/or can be readily determined via a calibration process. In other embodiments, the system 100 can include a different number of the cameras 112 and/or the cameras 112 can be positioned differently relative to another.

Referring to FIG. 1-3 together, in some aspects of the present technology the system 100 can generate a digitized view of the scene 108 that provides a user (e.g., a surgeon) with increased “volumetric intelligence” of the scene 108. For example, the digitized scene 108 can be presented to the user from the perspective, orientation, and/or viewpoint of their eyes such that they effectively view the scene 108 as though they were not viewing the digitized image (e.g., as though they were not wearing the head-mounted display 104). However, the digitized scene 108 permits the user to digitally rotate, zoom, crop, or otherwise enhance their view to, for example, facilitate a surgical workflow. Likewise, initial image data, such as CT scans and/or MRI data, can be registered to and overlaid over the image of the scene 108 to allow a surgeon to view these data sets together. Such a fused view can allow the surgeon to visualize aspects of a surgical site that may be obscured in the physical scene 108—such as regions of bone and/or tissue that have not been surgically exposed.

### II. Selected Embodiments of Determining Implant Position and Orientation Using Intraoperative Imaging Techniques and Models

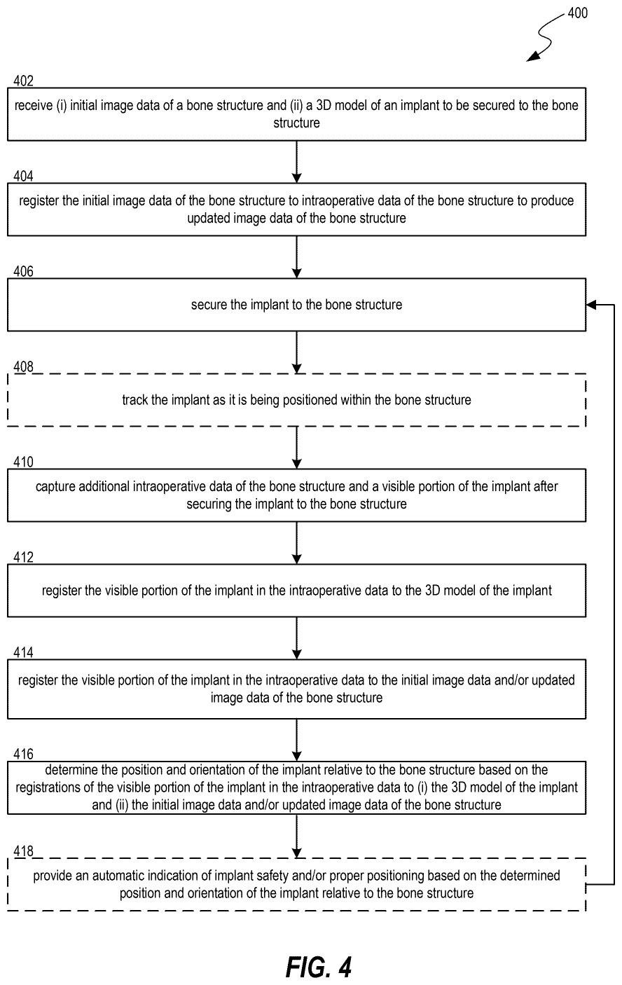

FIG. 4 is a flow diagram of a process or method 400 for determining the position and orientation of an implant within a bone structure in accordance with embodiments of the present technology. The method 400 can be carried out and/or performed by the system 100 of FIG. 1-3. Accordingly, although some features of the method 400 are described in the context of the system 100 shown in FIG. 1-3 for the sake of illustration, one skilled in the art will readily understand that the method 400 can be carried out using other suitable systems and/or devices described herein. Moreover, although reference is primarily made to determining the position and orientation of a pedicle screw in the spine of a patient undergoing spinal surgery, in other embodiments the method 400 can be carried out to determine the position and orientation for other types of implants relative to other bone structures in other types of surgical (e.g., orthopedic) procedures (e.g., hip, shoulder, ankle, wrist, elbow, digit, or knee arthroplasty; dental work; trauma procedure; bone resections and/or the like). Such implants can include interbody implants for placement in the femur, knee, tibia, hip, and/or the like. More generally, the present technology can be applied to help determine the final position and orientation of any surgical target (e.g., implant, bones, ligaments, flesh) relative to other surgical targets for which intraoperative data is available (as described below and with reference to FIG. 1-3).

At block 402, the method 400 can include receiving (i) initial image data of a bone structure and (ii) a three-dimensional (3D) model of an implant to be fixed/secured to/within the bone structure. As one example, the initial image data can be of all or a portion of the spine 309 (FIG. 3) and the 3D model can be a model of a pedicle screw to be implanted within the spine 309 during a surgical procedure. The 3D model can be a computer-aided design (CAD) model and/or other high-fidelity volumetric model. In some embodiments, the initial image data is preoperative image data. As described in detail above, the preoperative image data can be, for example, medical scan data representing a 3D volume of a patient, such as computerized tomography (CT) scan data, magnetic resonance imaging (MRI) scan data, ultrasound images, fluoroscopic images, and/or the like. In some embodiments, the initial image data is captured intraoperatively. For example, the initial image data can comprise 2D or 3D X-ray images, fluoroscopic images, CT images, MRI images, combinations thereof, and/or the like, that are captured of the patient within an operating room (e.g., immediately before a surgical procedure on the patient begins). In some embodiments, the initial image data comprises a point cloud, 3D mesh, and/or another 3D data set. In some embodiments, the initial image data comprises segmented 3D CT scan data of some or all of the spine 309 (e.g., segmented on a per-vertebra basis). In some embodiments, the initial image data can be image data, depth data, medical scan data, etc., that is captured intraoperatively such as, for example, just before the implant is secured within the bone structure. Accordingly, “initial image data” can comprise data captured at any point before the implant is secured within the bone structure.

At block 404, the method 400 can include registering the initial image data of the bone structure to intraoperative data of the bone structure. In some embodiments the intraoperative data includes real-time or near-real-time images of the spine 309 in the scene 108 captured by the cameras 112 and/or the depth cameras 118 of the sensor array 110. In some embodiments, the intraoperative image data includes (i) light field and/or RGB images from the cameras 112 and (ii) images from the depth cameras 118 that include encoded depth information about the scene 108. In addition to image data, the intraoperative data can include a depth map, such as a 3D point cloud or mesh generated from images from the cameras 112 and/or the depth sensor 118. Some spinal deformities can be large enough that they are not entirely visible to the cameras 112 within the scene 108. Accordingly, in some embodiments receiving the intraoperative image data includes receiving intraoperative image data from the sensor array 110 from different viewpoints relative to the scene 108 that capture the entire spinal deformity. For example, the camera array 110 can be moved (e.g., scanned) relative to the spine 309 to capture intraoperative data of the entire spine 309.

The initial image data and the intraoperative data initially exist in different coordinate systems such that the same features in both data sets are represented differently. Accordingly, at block 404, the method 400 can include registering the initial image data to the intraoperative data to, for example, establish a transform/mapping/transformation between the intraoperative data and the initial image data such that these data sets can be represented in the same coordinate system. In some embodiments, the registration process matches (i) 3D points in a point cloud or a 3D mesh representing the initial image data to (ii) 3D points in a point cloud or a 3D mesh of the intraoperative data. In some embodiments, the system 100 (e.g., the registration processing device 105) generates a 3D point cloud or mesh from intraoperative image data from the depth cameras 118 of the depth sensor 114, and registers the point cloud or mesh to the initial image data by detecting positions of fiducial markers and/or feature points visible in both data sets. For example, where the initial image data comprises CT scan data, rigid bodies of bone surface calculated from the CT scan data can be registered to the corresponding points/surfaces of the point cloud or mesh.

In some embodiments, the registration is based on/initiated by a surgeon or other user identifying corresponding points in both data sets. For example, the surgeon can identify points in the intraoperative image data that correspond to the same points in the initial image data, such as screw entry points identified by a preoperative plan. In some embodiments, the surgeon can identify the points by touching a tracked instrument to the spine 309. In other embodiments, the system 100 can employ other registration processes based on other methods of shape correspondence, and/or registration processes that do not rely on fiducial markers (e.g., markerless registration processes). For example, the registration/alignment process can utilize X-ray data, other medical imaging data, tracing information from an instrument moved through the scene 108, and/or other data sets. In some embodiments, the registration/alignment process can include features that are generally similar or identical to the registration/alignment processes disclosed in (i) U.S. patent application Ser. No. 16/749,963, titled “ALIGNING PREOPERATIVE SCAN IMAGES TO REAL-TIME OPERATIVE IMAGES FOR A MEDIATED-REALITY VIEW OF A SURGICAL SITE,” filed Jan. 22, 2020 and/or (ii) U.S. patent application Ser. No. 17/140,885, titled “METHODS AND SYSTEMS FOR REGISTERING PREOPERATIVE IMAGE DATA TO INTRAOPERATIVE IMAGE DATA OF A SCENE, SUCH AS A SURGICAL SCENE,” and filed Jan. 4, 2021, each of which is incorporated herein by reference in its entirety. In some embodiments, the registration can be carried out using any feature or surface matching registration method, such as iterative closest point (ICP), Coherent Point Drift (CPD), or algorithms based on probability density estimation like Gaussian Mixture Models (GMM).

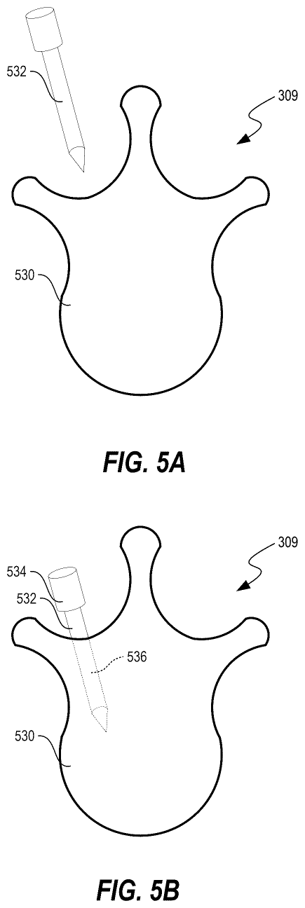

At block 406, the method 400 can include securing the implant to the bone structure. For example, a pedicle screw can be driven into a target vertebra of the spine 309. In some embodiments, the implant is positioned within the bone structure such that a first portion of the implant is within the bone structure and thereby visibly obscured from the sensor array 110 (and a surgical team carrying out the surgical procedure) while a second portion remains external to the bone structure or otherwise visible. For example, FIGS. 5A and 5B are side views of a vertebra 530 of the spine 309 before and after implantation of a pedicle screw 532 therein, respectively, in accordance with embodiments of the present technology. Referring to FIG. 5B, the pedicle screw 532 has been driven into the vertebra 530 (e.g., using a driver) such that the pedicle screw 532 includes a first portion 534 external to the vertebra 530 and that is visible to the sensor array 110 and/or a surgeon (e.g., to the naked eye of the surgeon), and a second portion 536 positioned within the vertebra 530 that is not visible to the sensor array 110.

Optionally, at block 408 the method 400 can include tracking the position of the implant as it is positioned within the bone structure. For example, the position of the implant can be tracked in one or more modalities (e.g., RGB images, light field images, infrared images) using the cameras 112, the trackers 113, and/or the depth sensor 114 of the sensor array 110.

At block 410, the method 400 can include capturing (and/or receiving) additional intraoperative data of the bone structure and a visible portion of the implant after securing the implant to the bone structure. The intraoperative data of the bone structure can include a portion of the bone structure adjacent to the implant. For example, referring to FIG. 5B, the intraoperative data can comprise one or more images of the vertebra 530 and the first portion 534 of the pedicle screw 532 that is positioned external to the vertebra 530. In some embodiments the intraoperative data includes real-time or near real-time images of the implant (e.g., the pedicle screw 532) and the bone structure (e.g., the vertebra 530) captured by the cameras 112 and/or the depth cameras 118 of the sensor array 110. Accordingly, the image data can include (i) light field and/or RGB images from the cameras 112 and/or (ii) images from the depth cameras 118 that include encoded depth information about the scene 108. In some embodiments, the intraoperative data include a depth map, such as a 3D point cloud, mesh, and/or another 3D data set generated from images from the cameras 112 and/or the depth sensor 118 that provides volumetric data about the bone structure and the visible portion of the implant. In some embodiments, capturing the intraoperative data includes capturing two or more images from the sensor array 110 from different viewpoints relative to the bone structure and the implant. For example, the sensor array 110 can be moved (e.g., scanned) relative to the vertebra 530 to capture images of the vertebra 530 and the first portion 534 of the pedicle screw 532 from different viewpoints. Additionally or alternatively, the images from different viewpoints can be generated by cameras positioned at different physical locations relative to one another.

At block 412, the method 400 can include registering the intraoperative data captured at block 410 of the visible portion of the implant to the 3D model of the implant. For example, the 3D model of the implant and the intraoperative data of the visible portion of the implant initially exist in different coordinate systems such that the same features in both data sets are represented differently. Accordingly, the method 400 can include registering the 3D model of the implant to the intraoperative data of the visible portion of the implant to, for example, establish a transform/mapping/transformation between the 3D model of the implant and the intraoperative data of the visible portion of the implant such that these data sets can be represented in the same coordinate system. In some embodiments, the registration process matches (i) 3D points in a point cloud or a 3D mesh representing the 3D model of the implant to (ii) 3D points in a point cloud or a 3D mesh of the intraoperative data representing the visible portion of the implant. In some embodiments, the system 100 (e.g., the registration processing device 105) registers the 3D model of the implant to the intraoperative data of the visible portion of the implant by detecting positions of fiducial markers and/or feature points visible in both data sets. For example, referring to FIG. 5, the first portion 534 of the pedicle screw 532 can comprise a screw head, and a head portion of the 3D model of the pedicle screw can be registered to the corresponding intraoperative data of the screw head 534 of the pedicle screw 532. In some embodiments, the registration is based on/initiated by a surgeon or other user identifying corresponding points in both data sets. For example, the surgeon can identify points in the 3D model of the implant that correspond to the same points in the intraoperative data of the visible portion of the implant. In some embodiments, the surgeon can identify the points by touching a tracked instrument to the visible portion of the implant (e.g., the first portion 534 of FIG. 5B). In other embodiments, the system 100 can employ other registration processes based on other methods of shape correspondence, and/or registration processes that do not rely on fiducial markers (e.g., markerless registration processes). In some embodiments, the registration can be carried out using any feature or surface matching registration method, such as iterative closest point (ICP), Coherent Point Drift (CPD), or algorithms based on probability density estimation like Gaussian Mixture Models (GMM).

At block 414, the method 400 can include registering the intraoperative data captured at block 410 of the visible portion of the implant to the initial image data of the bone structure. As described in detail above with reference to block 404, the initial image data can be registered to the intraoperative data of the bone structure before securing the implant to the bone structure. Accordingly, the visible portion of the implant in the intraoperative data can be registered to the initial image data by identifying/differentiating the visible portion of the implant in the intraoperative data relative to the bone structure in the intraoperative data. For example, referring to FIG. 5B, the position and orientation of the first portion 534 of the pedicle screw 532 in the intraoperative data can be compared to the surrounding intraoperative data of the vertebra 530 and registered to the initial image data of the vertebra 530 using the previous registration (block 404) between the intraoperative data of the bone structure and the initial image data.

At block 416, the method 400 can include determining a position and orientation of the implant relative to the bone structure based on the registrations of the visible portion of the implant in the intraoperative data to (i) the 3D model of the implant (block 412) and (ii) the initial image data of the bone structure (block 414). More specifically, these two registrations permit the system 100 (e.g., the processing device 102) to accurately relate/locate the 3D model of the implant to the initial image data based on the intraoperatively determined position of a visible portion of the implant. The 3D model of the implant provides (e.g., complete) volumetric data for the implant and the initial image data provides (e.g., complete) volumetric data for the bone structure. Thus, block 416 can include locating the 3D model of the implant relative to the initial image data in the same coordinate system based on the intraoperative data to determine a volumetric relationship and relative positioning between the implant and the bone structure. In particular, the system 100 can determine the position and orientation of the entire implant relative to the bone structure—including a portion of the implant that is positioned within and visibly obscured by the bone structure. For example, referring to FIG. 5B, the 3D model of the pedicle screw 532 can provide information about the position and orientation of the second portion 536 of the pedicle screw 532 that is not visible after implantation within the vertebra 530, after the 3D model of the pedicle screw 532 is registered to and/or overlaid on the initial image data of the vertebra 530.

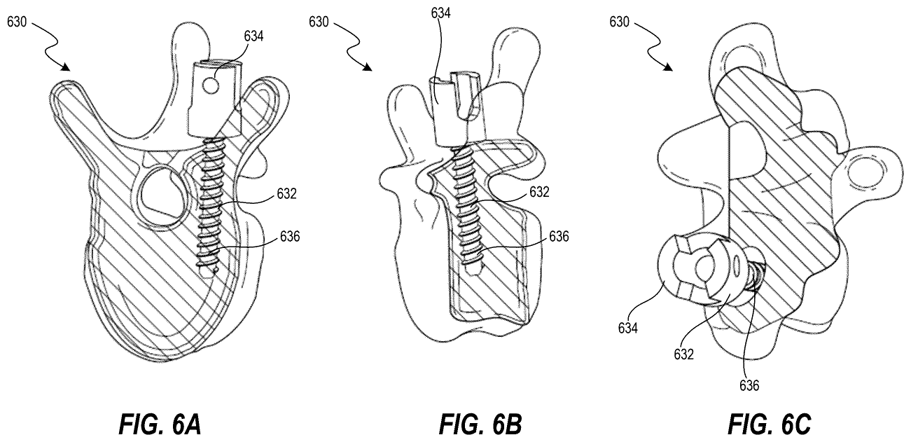

In some embodiments, the method 400 can include displaying a composite image (e.g., an overlay) of the 3D model of the implant and the initial image data of the bone structure. FIG. 6A-6C, for example, are an axial, sagittal, and coronal cutaway view, respectively, of initial image data 630 of the vertebra 530 of FIGS. 5A and 5B illustrating the overlay of a 3D model 632 of the pedicle screw 532 of FIGS. 5A and 5B in accordance with embodiments of the present technology. The combined display of the initial image data 630 and the 3D model 632 can be referred to as a composite image, composite rendering, composite display, and/or the like. Referring to FIG. 5A-6B together, the composite images shown in FIG. 6A-6C provide information about the position and orientation of the pedicle screw 532 including a first portion 634 of the 3D model 632 corresponding to the visible first portion 534 of the pedicle screw 532 and a second portion 636 corresponding to the obscured second portion 536 of the pedicle screw 532. In some embodiments, the system 100 can display the composite images shown in FIG. 6A-6C and/or other images in real-time or near real-time on the head-mounted display 104, the secondary display 226, and/or another display such that they are available to the surgeon and/or another user during the surgical procedure. In some aspects of the present technology, this can allow the surgeon to know in real-time or near real-time the position and orientation of the implant relative to the bone structure.

In some embodiments, implant tracking data obtained at block 408 can be used in any or all of blocks 412-416. For example, tracking data can be used to localize and/or otherwise inform the registrations at blocks 412 and 414. Likewise, tracking data can be used to validate the determined position and orientation at block 416 by defining a threshold region in which the implant is likely positioned within the bone structure.

At block 418, the method 400 can optionally include providing an automatic indication of implant safety and/or proper implant positioning. In some embodiments, the system 100 can determine whether the implant is properly/safely positioned relative to the bone structure and can generate an audible alarm, warning light, and/or other indicia if the position and/or orientation of the implant triggers a given condition. For example, the method 400 can generate an alarm if the determined position and orientation implant indicates that the implant is breaching the bone structure, is positioned within a threshold distance of a particular anatomical feature (e.g., within 1 mm of breaching through the wall of a vertebra), exhibits signs of movement (e.g., plough) etc. In some embodiments, the head-mounted display 104 and/or the secondary display 226 provide the warning light, audible alarm, and/or other indicia.

In some embodiments, after block 418, the method 400 can return to block 406 and be repeated for another implant to be implanted within the bone structure during the surgical procedure. Thus, each implant (e.g., screw or other fixation member) can be tracked continuously or near-continuously during the surgical procedure to provide an indication of the position and orientation of the implant relative to the bone structure, including the final position and orientation of a visibly-obscured (e.g., embedded) portion of the implant, to help a surgeon position and/or reposition the implant over the throughout the procedure. Thus, a surgeon does not have to rely on a post-implantation radiation-based imaging before continuing to position or correct the position of the implant.

In some embodiments, a similar process of capturing intraoperative data of the visible portion of the implant (e.g., block 410), registering the visible portion of the implant in the intraoperative data to the 3D model (e.g., block 412), registering the visible portion of the implant in the intraoperative to the initial image data of the bone structure (e.g., block 414), and determining the position and orientation of the implant relative to the bone structure based on the registrations of the visible portion of the implant in the intraoperative data to (i) the 3D model of the implant and (ii) the initial image data of the bone structure (e.g., block 416) can be repeated to assist with real-time or near-real-time plough detection. For example, a surgeon can determine that the 3D model of a given implant is moving in an undesired manner indicative of a loss of fixation of the implant's initial position with the bone structure as the implants are torqued/loaded during the surgical procedure. For example, such a method can determine that one or more pedicle screws are moving (e.g., ploughing) through a spine and/or have pulled out of the spine as rods are connected to the pedicle screws and loaded and/or as the spine is manipulated to achieve a desired alignment during the surgical procedure. More particularly, in some embodiments the method 400 includes determining an initial position and orientation of the implant relative to the bone structure, continuously monitoring the position and orientation of the implant over at least part of the course of the procedure (e.g., iterating through blocks 410-416 in real time or near real time), and providing an automatic indication (e.g., an alarm, light, and/or sound) if the position and orientation departs from the initial position and orientation by a threshold amount.

Additionally, in some embodiments blocks 410-416 of the method 400 can be carried out while the implant is secured to the bone structure. Accordingly, the method 400 can include determining the position and orientation of the implant relative to the bone structure during the implantation of the implant to provide, for example, a continuous (e.g., real time or near real time) indication of the position and orientation of the implant relative to the bone structure. In some such embodiments, the block 418 can be carried out to provide an automatic indication of implant safety and/or proper positioning. For example, the method 400 can generate an alarm if the determined position and orientation implant indicates that the implant is approaching or within a threshold distance of a particular anatomical feature (e.g., within 1 mm of breaching through the wall of a vertebra).

Although reference has primarily been made herein to determining the position and orientation of a pedicle screw in the spine of a patient undergoing spinal surgery, in other embodiments the methods of the present technology can be utilized to determine the position and orientation of other types of implants relative to other bone structures in other types of surgical procedures. More specifically, for example, the present technology is applicable to most orthopedic procedure that involve implants or bone resections in which a surgeon wants to know and document the final locations of the implants relative to bone they have been implanted into. For example, the present technology can be utilized in hip, shoulder, ankle, wrist, spine, elbow, digit, and/or knee arthroplasty procedures to restore the function of a joint. Specifically, the present technology can determine the position and orientation of an arthroplasty implant relative to the bone structure of the respective joint that is operated on. The present technology can also be utilized in trauma procedures that require implantation of a device to fix or hold a bone in a specific position relative to another part of the same bone or another bone of a patient. Additionally, the present technology can be utilized in dental work where a crown or implant is positioned relative to the jaw or other teeth of a patient. Likewise, the present technology can be utilized in bone resections where one or more bones are cut in a wedge shape to reposition it relative to itself or another bone, implants are used to hold the bones in a specific position, and the surgeon wants to know the final position of the bones relative to each other.

### III. Examples

The following examples are illustrative of several embodiments of the present technology:
1. A method of determining a position and orientation of an implant secured within a bone structure during a surgical procedure, the method comprising:
capturing intraoperative data of the implant and the bone structure while and/or after the implant is secured within the bone structure, wherein the intraoperative data includes a visible portion of the implant external to the bone structure;
registering the visible portion of the implant in the intraoperative data to a three-dimensional (3D) model of the implant;
registering the visible portion of the implant in the intraoperative data to initial image data of the bone structure; and
determining the position and orientation of the implant relative to the bone structure based on the registrations of the visible portion of the implant in the intraoperative data to (i) the 3D model of the implant and (ii) the initial image data of the bone structure.
2. The method of example 1 wherein the method further comprises generating a composite image including the initial image data of the bone structure and the 3D model overlaid on the initial image data at the determined position and orientation relative to the bone structure.
3. The method of example 2 wherein the method further comprises displaying the composite image on a display device.
4. The method of any one of examples 1-3 wherein the method further comprises continuously in real time or substantially real time—
capturing the intraoperative data;
registering the visible portion of the implant in the intraoperative data to the three-dimensional (3D) model of the implant;
registering the visible portion of the implant in the intraoperative data to the initial image data of the bone structure; and
determining the position and orientation of the implant relative to the bone structure based on the registrations of the visible portion of the implant in the intraoperative data to (i) the 3D model of the implant and (ii) the initial image data of the bone structure.
5. The method of example 4 wherein the method further comprises:
determining that the position and orientation of the implant relative to the bone structure has changed; and
generating an alert for a user based on determination.
6. The method of any one of examples 1-5 wherein the intraoperative data comprises depth data of the implant and the bone structure.
7. The method of any one of examples 1-6 wherein the intraoperative data comprises light field image data of the implant and the bone structure.
8. The method of any one of examples 1-7 wherein the method further comprises: positioning the implant at least partially within the bone structure; and
tracking the position of the implant as it is secured within the bone structure.
9. The method of any one of examples 1-8 wherein the method further comprises providing an automatic indication of proper or improper positioning of the implant relative to the bone structure based on the determined position and orientation of the implant.
10. The method of any one of examples 1-9 wherein capturing the intraoperative data includes capturing the intraoperative data with a sensor array having first cameras of a first type and second cameras of a second type, wherein the first type is different than the second type, and wherein the first cameras and the second cameras are fixedly mounted to a common frame.
11. The method of any one of examples 1-10 wherein the bone structure comprises a spine.
12. A method of determining a position and orientation of a spinal implant secured within a vertebra during a spinal surgical procedure, the method comprising:
capturing intraoperative data of the spinal implant and the vertebra while and/or after the spinal implant is secured within the vertebra, wherein the intraoperative data includes a visible portion of the spinal implant external to the vertebra;
registering the visible portion of the spinal implant in the intraoperative data to a three-dimensional (3D) model of the spinal implant;
registering the visible portion of the spinal implant in the intraoperative data to initial image data of the vertebra; and
determining the position and orientation of the spinal implant relative to the vertebra based on the registrations of the visible portion of the spinal implant in the intraoperative data to (i) the 3D model of the spinal implant and (ii) the initial image data of the vertebra.
13. The method of example 12 wherein the spinal implant comprises a pedicle screw.
14. The method of example 12 or example 13 wherein the method further comprises continuously in real time or substantially real time—
capturing the intraoperative data;
registering the visible portion of the spinal implant in the intraoperative data to a three-dimensional (3D) model of the spinal implant;
registering the visible portion of the spinal implant in the intraoperative data to initial image data of the vertebra; and
determining the position and orientation of the spinal implant relative to the vertebra based on the registrations of the visible portion of the spinal implant in the intraoperative data to (i) the 3D model of the spinal implant and (ii) the initial image data of the vertebra.
15. The method of any one of examples 12-14 the intraoperative data comprises depth data of the spinal implant and the vertebra, and wherein capturing the depth data includes capturing the intraoperative data with a depth sensor of a sensor array.
16. The method of example 15 wherein the intraoperative data further comprises light field image data of the spinal implant and the vertebra, and wherein capturing the light field image data includes capturing the light field image data with two or more cameras of the sensor array.
17. The method of example 16 wherein the two or more cameras and the depth sensor are fixedly mounted to a common frame.
18. The method of any one of examples 12-17 wherein the method does not comprise determining the position and orientation of the spinal implant relative to the vertebra with radiation-based imaging.
19. A system for determining a position and orientation of an implant secured within a bone structure during a surgical procedure, the system comprising:
a sensor array including multiple sensors fixed to a common frame, wherein the sensors are configured to capture intraoperative data of the implant and the bone structure while and/or after the implant is secured within the bone structure, wherein the intraoperative data includes a visible portion of the implant external to the bone structure; and
a processing device communicatively coupled to the sensor array, wherein the processing device is configured to—
   receive the intraoperative data of the implant and the bone structure from the sensor array;
   register the visible portion of the implant in the intraoperative data to a three-dimensional (3D) model of the implant;
   register the visible portion of the implant in the intraoperative data to initial image data of the bone structure; and
   determine the position and orientation of the implant relative to the bone structure based on the registrations of the visible portion of the implant in the intraoperative data to (i) the 3D model of the implant and (ii) the initial image data of the bone structure.
20. The method of example 19 wherein the multiple sensors include a depth sensor, and wherein the intraoperative data comprises depth data of the implant and the bone structure captured by the depth sensor.

### IV. Conclusion

The above detailed descriptions of embodiments of the technology are not intended to be exhaustive or to limit the technology to the precise form disclosed above. Although specific embodiments of, and examples for, the technology are described above for illustrative purposes, various equivalent modifications are possible within the scope of the technology as those skilled in the relevant art will recognize. For example, although steps are presented in a given order, alternative embodiments may perform steps in a different order. The various embodiments described herein may also be combined to provide further embodiments.

From the foregoing, it will be appreciated that specific embodiments of the technology have been described herein for purposes of illustration, but well-known structures and functions have not been shown or described in detail to avoid unnecessarily obscuring the description of the embodiments of the technology. Where the context permits, singular or plural terms may also include the plural or singular term, respectively.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/We claim:

1. A method of determining a position and orientation of an implant secured within a bone structure during a surgical procedure, the method comprising:
capturing intraoperative data of the implant and the bone structure while and/or after the implant is secured within the bone structure, wherein the intraoperative data includes a visible portion of the implant external to the bone structure;
registering the visible portion of the implant in the intraoperative data to a three-dimensional (3D) model of the implant;
registering the visible portion of the implant in the intraoperative data to initial image data of the bone structure; and
determining the position and orientation of the implant relative to the bone structure based on the registrations of the visible portion of the implant in the intraoperative data to (i) the 3D model of the implant and (ii) the initial image data of the bone structure.

2. The method of claim 1, wherein the method further comprises generating a composite image including the initial image data of the bone structure and the 3D model overlaid on the initial image data at the determined position and orientation relative to the bone structure.

3. The method of claim 2, wherein the method further comprises displaying the composite image on a display device.

4. The method of claim 1, wherein the method further comprises continuously in real time or substantially real time—
capturing the intraoperative data;
registering the visible portion of the implant in the intraoperative data to the three-dimensional (3D) model of the implant;
registering the visible portion of the implant in the intraoperative data to the initial image data of the bone structure; and
determining the position and orientation of the implant relative to the bone structure based on the registrations of the visible portion of the implant in the intraoperative data to (i) the 3D model of the implant and (ii) the initial image data of the bone structure.

5. The method of claim 4 wherein the method further comprises:
determining that the position and orientation of the implant relative to the bone structure has changed; and
generating an alert for a user based on determination.

6. The method of claim 1, wherein the intraoperative data comprises depth data of the implant and the bone structure.

7. The method of claim 1, wherein the intraoperative data comprises light field image data of the implant and the bone structure.

8. The method of claim 1, wherein the method further comprises:
positioning the implant at least partially within the bone structure; and
tracking the position of the implant as it is secured within the bone structure.

9. The method of claim 1, wherein the method further comprises providing an automatic indication of proper or improper positioning of the implant relative to the bone structure based on the determined position and orientation of the implant.

10. The method of claim 1, wherein capturing the intraoperative data includes capturing the intraoperative data with a sensor array having first cameras of a first type and second cameras of a second type, wherein the first type is different than the second type, and wherein the first cameras and the second cameras are fixedly mounted to a common frame.

11. The method of claim 1, wherein the bone structure comprises a spine.

12. A method of determining a position and orientation of a spinal implant secured within a vertebra during a spinal surgical procedure, the method comprising:
capturing intraoperative data of the spinal implant and the vertebra while and/or after the spinal implant is secured within the vertebra, wherein the intraoperative data includes a visible portion of the spinal implant external to the vertebra;
registering the visible portion of the spinal implant in the intraoperative data to a three-dimensional (3D) model of the spinal implant;
registering the visible portion of the spinal implant in the intraoperative data to initial image data of the vertebra; and
determining the position and orientation of the spinal implant relative to the vertebra based on the registrations of the visible portion of the spinal implant in the intraoperative data to (i) the 3D model of the spinal implant and (ii) the initial image data of the vertebra.

13. The method of claim 12, wherein the spinal implant comprises a pedicle screw.

14. The method of claim 12, wherein the method further comprises continuously in real time or substantially real time—
capturing the intraoperative data;
registering the visible portion of the spinal implant in the intraoperative data to a three-dimensional (3D) model of the spinal implant;
registering the visible portion of the spinal implant in the intraoperative data to initial image data of the vertebra; and
determining the position and orientation of the spinal implant relative to the vertebra based on the registrations of the visible portion of the spinal implant in the intraoperative data to (i) the 3D model of the spinal implant and (ii) the initial image data of the vertebra.

15. The method of claim 12 wherein the intraoperative data comprises depth data of the spinal implant and the vertebra, and wherein capturing the depth data includes capturing the intraoperative data with a depth sensor of a sensor array.

16. The method of claim 15, wherein the intraoperative data further comprises light field image data of the spinal implant and the vertebra, and wherein capturing the light field image data includes capturing the light field image data with two or more cameras of the sensor array.

17. The method of claim 16, wherein the two or more cameras and the depth sensor are fixedly mounted to a common frame.

18. The method of claim 12, wherein the method does not comprise determining the position and orientation of the spinal implant relative to the vertebra with radiation-based imaging.

19. A system for determining a position and orientation of an implant secured within a bone structure during a surgical procedure, the system comprising:
a sensor array including multiple sensors fixed to a common frame, wherein the sensors are configured to capture intraoperative data of the implant and the bone structure while and/or after the implant is secured within the bone structure, wherein the intraoperative data includes a visible portion of the implant external to the bone structure; and
a processing device communicatively coupled to the sensor array, wherein the processing device is configured to—
receive the intraoperative data of the implant and the bone structure from the sensor array;
register the visible portion of the implant in the intraoperative data to a three-dimensional (3D) model of the implant;
register the visible portion of the implant in the intraoperative data to initial image data of the bone structure; and
determine the position and orientation of the implant relative to the bone structure based on the registrations of the visible portion of the implant in the intraoperative data to (i) the 3D model of the implant and (ii) the initial image data of the bone structure.

20. The system of claim 19, wherein the multiple sensors include a depth sensor, and wherein the intraoperative data comprises depth data of the implant and the bone structure captured by the depth sensor.
